(19) United States
(12) Patent Application Publication
(10) Pub. No.: US 2012/0143096 A1
(43) Pub. Date: Jun. 7, 2012

(54) Cosmetic method for promoting recovery of skin barrier function using sound wave having a specific frequency

(75) Inventors: Mitsuhiro Denda, Yokohama-shi (JP); Masashi Nakatani, Yokohama-shi (JP)

(73) Assignee: Shiseido Company, Ltd.

(21) Appl. No.: 12/926,694

(22) Filed: Dec. 3, 2010

(51) Int. Cl. A61N 7/00

(52) U.S. Cl. 601/2

(57) ABSTRACT

The present invention provides a cosmetic method for promoting the recovery of skin barrier function, comprising applying a sound wave having a frequency of 10,000 to 50,000 Hz to the skin.

### TECHNICAL FIELD

The present invention relates to a cosmetic method for promoting the recovery of skin barrier function using a sound wave having a specific frequency, and a sound wave generator for using the method.

### BACKGROUND ART

Skin damaged due to various dermatological disorders such as atopic dermatitis, psoriasis, and contact dermatitis, etc., is known to lose its moisture much more than normal skin. The increase of so-called transepidermal water loss (TEWL) has been considered to be associated with the reduction of a component which is considered to have a function of maintaining moisture or serving as a barrier in the skin.

It has been reported that skin function decreases in accordance with the decrease of a skin barrier function, resulting in a skin proliferative disorder, etc. Especially, the elderly require a long time to restore the decreased skin barrier function, thus, many agents for promoting the recovery of skin barrier function, which is effective in preventing skin proliferative disorders, etc., due to a decrease of the skin function by aging, have been developed.

It has been commonly accepted for many years that peripheral nerve terminals exist in the dermis of the skin, and the peripheral nervous system has an important role in a sensory reception system which responds to external physical or chemical stimuli. However, over the last decade, a series of sensory receptor proteins which respond to specific temperatures, osmotic pressures, electrical potentials, mechanical stresses, etc., have been identified not only in the peripheral nervous system but also in epidermal keratinocytes (Dhaka A, et al., “TRP ion channels and temperature sensation”, Annu Rev Neurosci 29, pp. 135-161, 2006; and Denda M, et al., “Effects of skin surface temperature on epidermal permeability barrier homeostasis”, J Invest Dermatol, 127, pp. 654-659, 2007).

Furthermore, a previous study by the inventors of the present invention verified the effect of a specific temperature or visible light on skin barrier homeostasis (Denda M, et al., “Epidermal keratinocytes as the forefront of the sensory system”, Exp Dermatol 16, pp. 157-161, 2007). Receptor proteins activated by such stimuli are also expressed in epidermal keratinocytes (Dhaka A et al., forecited; and Tsutsumi M, et al., “Expressions of rod and cone photoreceptor-like proteins in human epidermis”, Exp Dermatol, Published Online: Mar. 12, 2009). The above-mentioned experimental results indicate that epidermal keratinocytes have a sensory receptor system similar to a peripheral nervous system.

### SUMMARY OF INVENTION

Sound is perceived when the vibration of an object provides stimulation to acoustic cells through air, etc. The range of frequencies of sound which can be perceived by humans (audible range) varies depending on age, gender, etc. Although the audible range of humans varies among individuals as mentioned above, the range is generally considered to be from 20 Hz to 16,000 Hz. (e.g., Heffner R S., “Primate hearing from a mammalian perspective”, Anat Rec A Discov Mol Cell Evol Biol. 2004, November; 281(1), pp. 1111-1122). On the other hand, sound waves having a frequency of more than 20,000 Hz generally cannot be perceived by human ears, and are defined as ultrasonic waves.

A study by Oohashi et al. has revealed that an ultrasonic wave exerts its effect on brain wave and systemic hormone level of human without going through auditory stimulation (Oohashi T, et al., “Inaudible high-frequency sounds affect brain activity: hypersonic effect”, J. Neurophysiol. 2000, June, 83(6), pp. 3548-58; Yagi R, et al., “Modulatory effect of inaudible high-frequency sounds on human acoustic perception”, Beuroschi Lett. 2003 Nov. 20, 351(3), pp. 191-195; and Kawai N, et al., “Catecholamines and opoid peptides increase in plasma in humans during possession trances”, Neuroreport. 2001, Nov. 16, 12(16), pp. 3419-3423). These facts indicate that an unknown sensory system which perceives ultrasonic waves exists on human skin. However, the influence of sound on the homeostasis of skin barrier function has not been studied.

The inventors of the present invention evaluated the influence of sound on the homeostasis of a skin barrier on the basis of the hypothesis that not only an ultrasonic wave but also a sound wave of an audible range can be sensed on epidermis. As a result, the inventors of the present invention found that a sound wave having a specific frequency has an effect of promoting the recovery of skin barrier function, and achieved the present invention.

More specifically, the present application includes the following inventions:

[1] A cosmetic method for promoting the recovery of skin barrier function, comprising applying a sound wave having a frequency of 10,000 to 50,000 Hz to the skin.
[2] A method of [1] wherein the sound wave is applied at a sound pressure level of 60 to 200 db.
[3] A sound wave generator for promoting the recovery of skin barrier function, comprising an oscillator which generates a sound wave having a frequency of 10,000 to 50,000 Hz, a function generator for regulating the frequency of the sound wave, and a horn to transmit the sound wave to a skin.
[4] A sound wave generator of [3], further comprising a means to regulate the sound pressure level of the sound wave to no less than 60 db.

An ultrasonic wave having a frequency of 15 MHz provides the epidermis with water and ions, and as a result, the ultrasonic wave changes the concentration gradient of calcium ion and the secretion of lamellar body, according to the study of Menon et al. (Menon GK, et al., “Selective Obliteration of the Epidermal Calcium Gradient Leads to Enhanced Lamellar Body Secretion”, Journal of Investigative Dermatology (1994) 102, pp. 789-795). On the other hand, the influence of a sound wave having a frequency of 10,000 to 50,000, far less than that of ultrasonic therapy, especially for promoting the recovery of skin barrier function, was conventionally unknown. It is not our intention to be bound by the theory, but sound is considered to exert its influence on the phase transition between a lamellar body and a cell membrane.

As mentioned above, the present invention provides a novel cosmetic method useful in clinical dermatology, in order to recover the skin barrier function. Also, a sound wave generator provided by the invention of the present application may comprise a universal device such as audio equipment, not a specialized device required for conventional ultrasonic therapy.

### BRIEF DESCRIPTION OF DRAWINGS

FIG. 1 represents a graph showing the effect of promoting the recovery of the skin barrier function when a sound wave having a specific frequency is applied on a tape-stripped skin. C of the horizontal axis indicates the control.

FIG. 2 represents a graph explaining an influence of the distance between the skin and the sound source on an effect of promoting the recovery of the skin barrier function. C of the horizontal axis indicates the control.

FIG. 3 represents a graph explaining an influence of the sound pressure level on the effect of promoting the recovery of the skin barrier function. C of the horizontal axis indicates the control.

FIG. 4 represents a graph explaining an effect of a sound wave on promoting the recovery of the skin barrier function when a TRPA1 antagonist, HC030031, is preliminarily applied on, the skin. C of the horizontal axis indicates the control, and +HC indicates that the preliminary treatment with HC030031 is conducted.

### DESCRIPTION OF EMBODIMENTS

In the first aspect of the present invention, a cosmetic method for promoting the recovery of the skin barrier function, comprising applying a sound wave having a frequency of 10,000 to 50,000 Hz to the skin is provided.

A sound wave is an elastic wave having an audible frequency, and is referred to as sound from the audiological viewpoint (“Rikagaku jiten” 4th edition, Iwanami Shoten). In this specification, for the sake of convenience, the term “sound wave” is used, as a collective term for all elastic waves propagating an elastic body, to mean waves of not only audible frequencies but also inaudible frequencies.

The unit of frequency is represented by hertz (Hz), which corresponds to the frequency of vibration per second. The present invention promotes the recovery of skin barrier function by applying a high frequency sound wave having a vibration frequency of no less than 10,000 per second to the skin. A cosmetic method of the present invention can be conducted using a sound wave generator comprising a device for regulating frequencies, e.g., a function generator. A sound wave having a frequency of no less than 20,000 Hz is generally referred to as an ultrasonic wave, but a sound wave having a frequency of around 20,000 Hz can be generated by audio equipment depending on its performance.

The sound wave used in the present invention has a frequency of 10,000 to 50,000 Hz, preferably 10,000 to 30,000 Hz. However, the frequency is arbitrarily modified depending on the conditions, etc., of the skin on which the sound wave is applied.

The cosmetic method of the present invention can be conducted by placing the skin, in which the promotion of the recovery of skin barrier function is intended, in front of a sound wave generator, e.g., a speaker connected to audio equipment, and then exposing the skin to sound having a specific frequency generated by the generator during a specific period of time. The cosmetic method of the present invention is preferably conducted while the sound wave generator, especially the horn moiety of the generator, is in contact with skin. However, the sound wave generator can be place away from the skin at a specific distance, e.g., 1 to 3 cm.

When a sound wave is applied to the skin distant from a sound source, the amplitude of a sound wave is reduced in proportion to the distance (attenuation). In this case, the sound pressure level (db) of a sound wave employed may be intensified in order to compensate for the attenuation effect. In a cosmetic method of the present invention, the sound pressure level can be regulated to be no less than 60 db, e.g., in the range of 70 to 200 db. Because the recovery of the skin barrier function is promoted further in accordance with the increase of sound pressure level, the sound pressure level employable in the present invention is not limited to the above-mentioned range. The upper limit of the range varies depending on the performance of the generator employed.

The term “skin barrier function” used in the present specification means the retention of moisture in the body and the prevention of invasion of viruses and bacteria, etc., due to the skin, especially, epidermis. The function can be evaluated by measuring transepidermal water loss (TEWL) (unit: g/m²·h) under a non-sweat condition.

Also, in the present invention, the term “promoting the recovery of skin barrier function” means having an effect of promoting a TEWL recovery rate, namely, means that the TEWL value at each measuring time clearly shows a significant difference compared to a control when the TEWL value of the skin immediately after the tape stripping is 0% and the TEWL value before the tape stripping is 100%. The term is different from the so-called preventive effect of improving damaged skin according to the method by Andrew et al. (J Invest Dermatol, 86; 598, 1986) wherein a judgment is made after treating skin with a cotton ball having a 4% sodium dodecyl, sulfate (SDS) aqueous solution soaked therein.

Examples of skin requiring the recovery of the skin barrier function include: skin having a decreased skin barrier function due to skin disorders, various stresses, and skin problems, etc.; skin having a low skin barrier function due to the incomplete formation of a skin barrier layer in the case of transplanted skin; and skin having a decreased skin barrier function due to transplantation. Therefore, the evaluation method of the present invention includes not only an evaluation of the recovery of a decreased skin barrier function but also an evaluation of a substance which can improve the function of the skin having a low skin barrier function. The subject may be mammalian, especially human skin, to which the substance evaluated to be effective in promoting the recovery of skin barrier function of the present invention is applied.

Furthermore, from the perspective of preventing skin proliferative disorders, etc., due to the decrease of skin function by aging as mentioned above, it is considered that the above-mentioned sound wave can be applied to the skin of elderly persons. The sound wave can also be applied on artificial skin. This is because a skin barrier layer of the artificial skin, which is obtained by cultivating skin cells, etc., isolated from a living organism, is sometimes incompletely formed, and thereby the skin barrier function thereof is generally low.

In the second aspect, the present invention provides a sound wave generator for promoting the recovery of skin barrier function, comprising an oscillator which generates a sound wave having a frequency of 10,000 to 50,000 Hz, a function generator for regulating the frequency of the sound wave, and a horn to transmit the sound wave to the skin.

As the oscillator, any oscillator which generates a sound wave having a frequency in the range of 10,000 to 50,000 Hz is employable. Preferably, the oscillator generates a sound wave having a frequency of 10,000 to 30,000 Hz. The frequency can be regulated by a universal function generator. The sound wave generated by the oscillator is applied to the skin through a horn. Audio equipment comprising a speaker which plays a role as an oscillator and a horn, and an amplifier which amplifies the sound wave generated from the oscillator is preferable as a sound wave generator of the invention of the present application. As described above, in the present invention, a device in which a commercially available function generator and audio equipment are connected to each other can be used as a sound wave generator. In a preferred embodiment, a sound wave generator of the present invention comprises a means to regulate the sound pressure level of a sound wave. The sound pressure level can be regulated to be no less than 60 db, e.g., in the range of 70 to 200 db. When audio equipment is employed, audio equipment comprising an amplifier having a function to regulate the sound pressure level is preferable.

The horn can be arbitrarily changed depending on the shape of the skin on which the sound wave is applied. For example, when the sound wave is applied to the skin on an arm, the horn can be formed in a cylindrical form.

### EXAMPLES

Next, the invention of the present application will be specifically explained with reference to the following examples.

### Study of Effect of Promoting Recovery of Skin Barrier Function

In the following experiments, effects of promoting the recovery of skin barrier function when sound waves having various frequency ranges and sound pressure levels are applied to the skin were evaluated. In the present testing, a multi-channel integrated amplifier (TA-DA3400ES, manufactured by Sony, Japan) connected with both a function generator (Type 19, manufactured by Wavelike, USA, TX, Ft Worth) and a full-range speaker FE 87E (manufactured by Fostex, Japan) was used as a sound wave generator. The sound pressure level was evaluated using a precision sound level meter NA-28 (manufactured by Rion, Japan).

Regarding the effect of promoting the recovery of skin barrier function, the skin barrier function was destroyed by applying tape-stripping on the skin of a hairless mouse, and the process of the recovery of the destroyed skin barrier function to the original condition was evaluated using the transepidermal water loss (TEWL) as an indicator (Type HR-1, HOSHINO, Japan) as follows.

1. Transepidermal water loss (TEWL) in the vicinity of the back of a hairless mouse was measured by a water loss measuring device MEECO (manufactured by MEECO, Warrington, Pa., USA). This value was defined as a TEWL recovery rate of 100%.
2. The skin barrier was destroyed by removing the horny layer of epidermis of a hairless mouse using adhesive tape. This manipulation was repeated until the TEWL value became about 800 to 900. The value obtained by subtracting the measured value of the horny layer before the removal from the measured value of the horny layer after the removal was defined as the value of the most damaged state, i.e., a recovery rate of 0%.
3. When a specific period of time (1, 3, 6, or 24 hours) had passed after the removal of the horny layer, a sound wave of 5, 10, 20, or 30 kHz was applied on the horny layer by contacting the skin with the speaker. The skin which was not exposed to a sound wave was defined as the control.
4. After 1 hour, the application of the sound wave was stopped and TEWL was measured by MEECO. The recovery rate was calculated by subtracting the TEWL value before the removal of the horny layer from the value measured at each period of time, in the same manner as the removal of the horny layer.
More specifically, the recovery rate was calculated according to the following equation.

Recovery rate(%)=((TEWL at each time after the removal of the horny layer)−(TEWL before the removal of the horny layer))/((TEWL after the removal of the horny layer)−(TEWL before the removal of the horny layer))×100

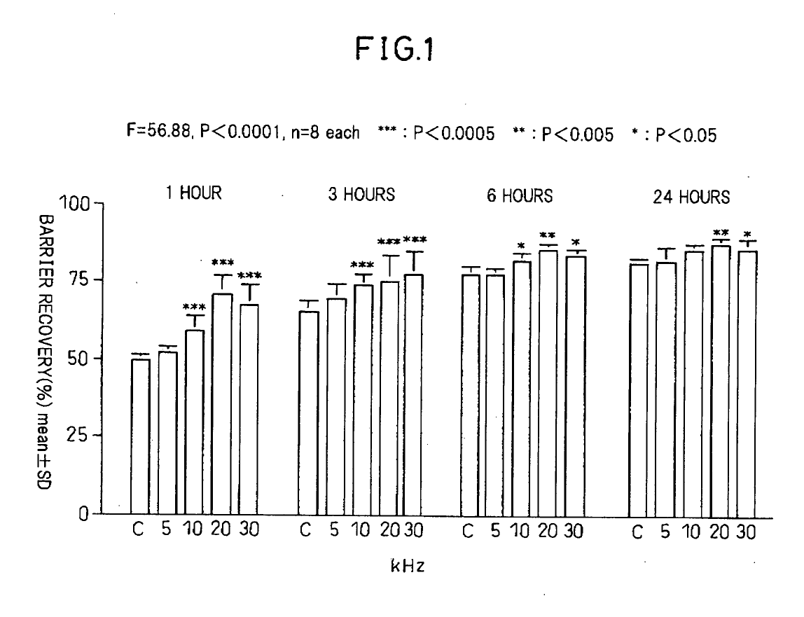

FIG. 1 shows the recovery rate (%) of skin barrier function measured by the testing. As clearly shown in FIG. 1, a sound wave having a frequency of 10 to 30 kHz significantly promoted the recovery of skin barrier function at any period of time measured. On the other hand, there was no significant difference between the results of a sound wave having a frequency of 5 kHz and the control. The sound pressure level in the present study was 83 db.

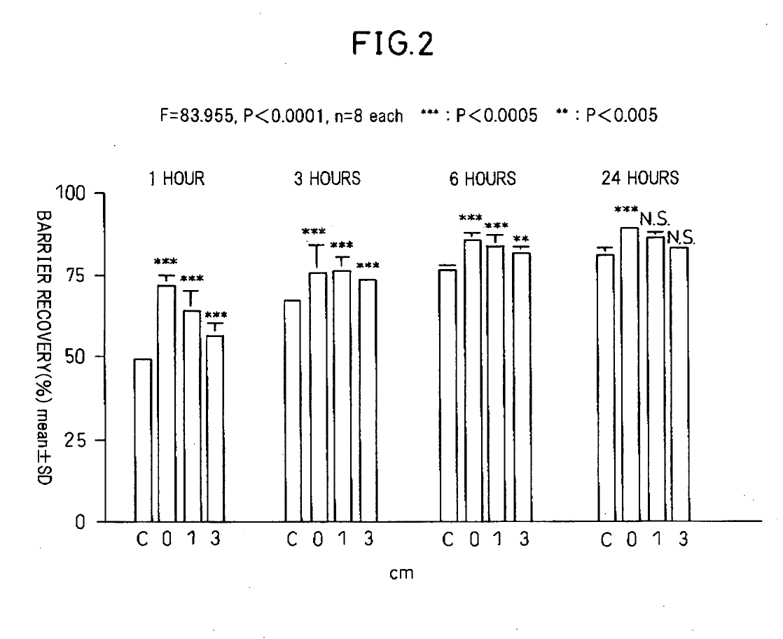

Subsequently, the skin was moved away from the speaker, and it was confirmed that the effect of promoting the recovery was actually attributed to the sound wave. When the speaker was 1 cm and 3 cm from the skin, the effect of promoting the recovery of skin barrier function was significantly increased compared to the control (FIG. 2). The sound pressure at each distance was 83 db at 0 cm, 78 db at 1 cm, or 70 db at 3 cm.

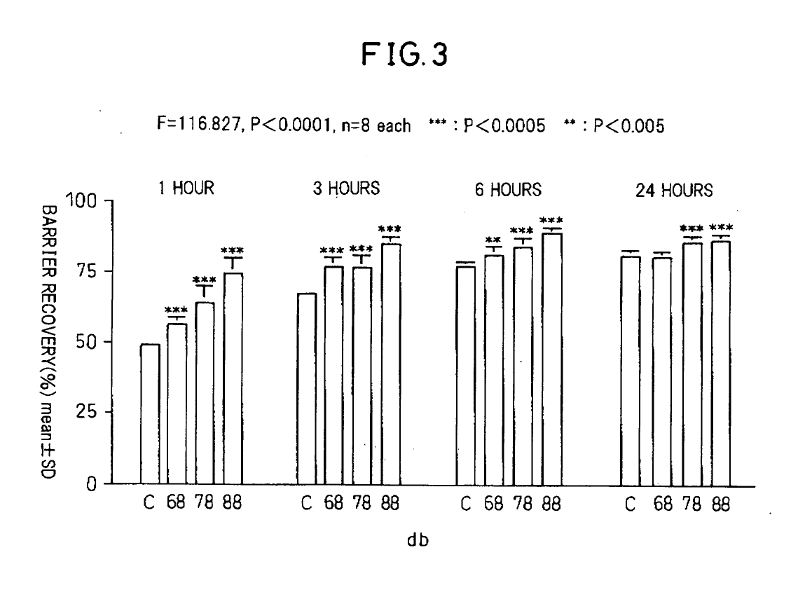

Furthermore, the influence of the sound pressure level for promoting the recovery of skin barrier function was studied. In this study, the above-mentioned experimental procedure was conducted using a sound wave having a frequency fixed at 20,000 Hz, and three kinds of sound pressure levels of 68, 78, and 88 db. As a result of these studies, the recovery rate increased in accordance with the increase of the sound pressure level (FIG. 3).

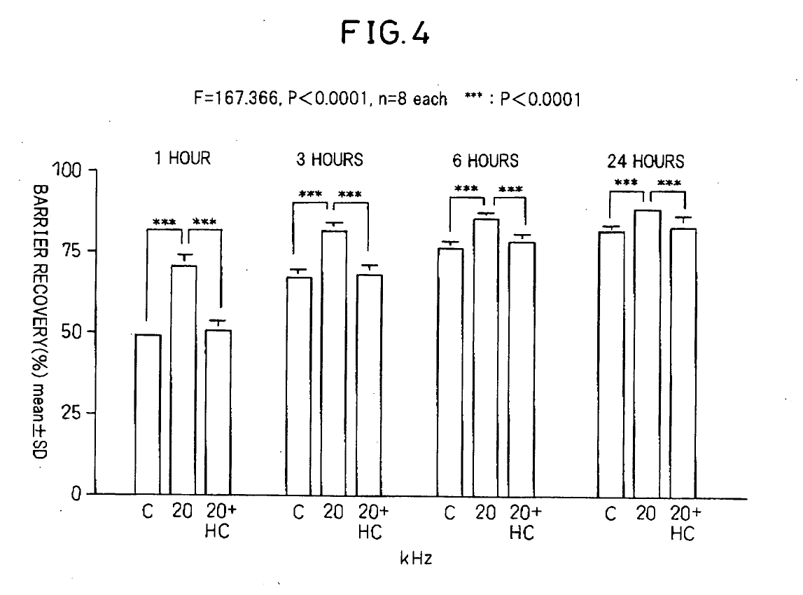

According to a study by Corey et al., TRPA1 expressed in hair cells in the inner ear of vertebrates is related to the auditory sense (Corey D P, et al., “TRPA1 is a candidate for the mechnosensitive transduction channel of vertebrate hair cells”, Nature, Dec. 9, 2004; 432 (7018): 723-30). Thus, we examined the effect of a TRPA1 antagonist for promoting the recovery of skin barrier function due to a sound wave. The skin was treated with the TRPA1 antagonist, HC030031, before applying a sound wave, and the experiment explained as above was conducted. As a result, the effect which promotes the recovery of skin barrier function due to a sound wave was inhibited (FIG. 4). Also, Ruzanna Atoyan et al. confirmed an immune response of TRPA1 antibody on a mouse epidermis (Non-Neuronal Expression of Transient Receptor Potential Type A1 (TRPA1) in Human Skin, J Invest Dermatol advance online publication, Mar. 12, 2009). These results indicate that a system that perceives sound exists on epidermis.

It is unclear as to how sound acts on the recovery of skin barrier function. However, the results mentioned above clearly show that a sound wave having a far lower frequency than the frequencies used in a conventional ultrasonic wave therapy contributes to promoting the recovery of skin barrier function. Therefore, according to the present invention, the recovery of skin barrier function can be promoted using commercially available audio equipment, without using a special ultrasonic wave generator.

1. A cosmetic method for promoting the recovery of skin barrier function, comprising applying a sound wave having a frequency of 10,000 to 50,000 Hz to the skin.

2. A method of claim 1, wherein the sound wave is applied at a sound pressure level of 60 to 200 db.

3. A sound wave generator for promoting the recovery of skin barrier function, comprising an oscillator which generates a sound wave having a frequency of 10,000 to 50,000 Hz, a function generator for regulating the frequency of the sound wave, and a horn to transmit the sound wave to the skin.

4. A sound wave generator of claim 3, further comprising a means to regulate the sound pressure level of the sound wave to no less than 60 db.
